(19) United States
(12) Patent Application Publication
(10) Pub. No.: US 2026/0207919 A1
(43) Pub. Date: Jul. 23, 2026

(54) BIVENTRICULAR ASSIST SYSTEM

(71) Applicants: ISTANBUL BILGI UNIVERSITESI, Istanbul (TR); KOC UNIVERSITESI, Istanbul (TR)

(72) Inventors: Ibrahim Basar AKA, Istanbul (TR); Kerem PEKKAN, Istanbul (TR); Canberk YILDIRIM, Istanbul (TR)

(73) Assignees: ISTANBUL BILGI UNIVERSITESI, Istanbul (TR); KOC UNIVERSITESI, Istanbul (TR)

(21) Appl. No.: 18/730,345

(22) PCT Filed: Jan. 19, 2022
(86) PCT No.: PCT/TR2022/050037

§ 371 Date: Jul. 19, 2024

(51) Int. Cl. A61M 60/183; A61M 60/232; A61M 60/523; A61M 60/882

(52) U.S. Cl. CPC A61M 60/183; A61M 60/232; A61M 60/523; A61M 60/882; A61M 2207/00

(57) ABSTRACT

A biventricular assist system includes a left ventricle assist device and a self-driving integrated aortic turbine ventricle assist device including a blood pump and blood turbine coupled together that can be easily manufactured to patient specifications. A control method using a master/slave approach is provided, wherein said biventricular assist system able to mimic the Frank-Starling mechanism of the heart without need for external sensors or additional mechanisms.

### CROSS REFERENCE TO THE RELATED APPLICATIONS

This application is the national phase entry of International Application No. PCT/TR2022/050037, filed on Jan. 19, 2022, the entire contents of which are incorporated herein by reference.

### TECHNICAL FIELD

The present invention relates to a biventricular mechanical assist system for patients with right heart failure. More specifically, the invention relates to a biventricular assist system including a left ventricle assist device and a self-driving integrated aortic turbine ventricle assist device that can be customized and easily manufactured to patient specifications and is able to mimic the natural Frank-Starling mechanism of the heart without the need for external sensors, feedback or additional control mechanisms.

### BACKGROUND

Today, bridge to heart transplant therapy is commonly conducted by mechanical ventricle assist device (VAD) implantation. Currently, there are several designs for VADs, which can be classified into four areas. Left and right ventricle assist devices for adults, left and right ventricle assist devices for children, short-term emergency support systems (such as impella and intra-aortic balloon pump) and single-ventricle right side support systems for Fontan patients. Among these, the most commonly used devices are left ventricle assist devices (LVAD).

However, right ventricular failure (RVF) or right heart failure (RHF) due to failure to adjust LVAD speed in accordance with the venous return is an increasingly common clinical complication at the perioperative stage of LVAD implantation and a major cause of postoperative morbidity and mortality. RHF leads to deprived filling of the left ventricle, and thus, low LVAD output or suction events. Suction occurs when the pump tries to draw more blood than is available in the ventricle due to high pump speed and can result in reduced forward flow of blood, hence inducing ischemia on the heart as well as distal organs, hemolysis, release of ventricular thrombus leading to stroke and tissue damage at VAD inlet. Patients with severe right ventricle failure may require prolonged inotropes, and persistent elevation of the central venous pressure may lead to liver dysfunction and sometimes multiple organ failure.

The Frank-Starling mechanism is the means by which the heart increases its cardiac output when the blood volume returning to it increases. The greater the blood volume in the heart (end-diastolic volume), the more the cardiac muscles are stretched; and the heart exerts more force in pumping blood when it is filled with a greater volume. This mechanism is diminished in case of heart failure due to weaker ventricles and in VADs due to the difficulty in adjusting VAD speed in accordance with the venous return. Various physiological control systems for continuous and pulsatile flow VADs have been developed to prevent suction by monitoring pump current and pressure variation. These controllers target the highest possible pump speed without a suction event occurring. A control system for VADs must be safe and adaptable. Namely, severe suction must be avoided, protecting the heart muscle, and the control system must be able to automatically adjust pump speed according to the patient's level of activity, such as changes in posture, exercise, straining or in response to intercurrent illness. There have been attempts in the art to provide Frank-Starling-like control systems for single VADs, as exemplified in US 2020/147284, EP 2 988 795, EP 3 423 125, and U.S. Pat. No. 7,476,200.

However, these systems may not be able to prevent RHF, and patients with end-stage RHF may still require biventricular assist devices (BiVAD). Currently available BiVADs generally require the use of two devices, an LVAD and an RVAD, with separate controllers, which can cause left and right outflow control issues. It is possible to use two separate self-regulating Frank-Starling systems, however, this results in long settling times and possibly unstable operation between the left and right pumps. As variations in one pump speed require conforming adjustments in the other pump, the ability to maintain a balance between the left and right outflow of a BiVAD system is essential for successful device operation.

The attempts made in the state of the art to address these issues are described below.

EP 3 423 125 discloses a biomedical device including a blood pump, pressure sensors placed within the circulatory system of the patient and including at least two different preset control algorithms for regulating the operating point of the blood pump based on the preload values determined from pressure measurements.

U.S. Pat. No. 8,657,875 discloses an artificial heart device including a first rotary pump having an input to receive blood and an output to provide blood to a patient's lungs (RVAD) and a second rotary pump having an input to receive blood and an output to provide blood to the patient's body (LVAD). The device also includes a first sensor associated with the RVAD, a second sensor associated with the LVAD, wherein said sensors are pressure and/or flowrate sensors, and a control system coupled to the first sensor, the second sensor, the VADs and configured to control characteristics of the VADs based on signals received from the sensors.

Stevens and colleagues disclose a dual rotary LVAD system for biventricular support, wherein one pump's controller is dependent on the other, using a master/slave (MS) approach. The MS control system is implemented by designating one pump (master) to be responsible for total flow variation and the other (slave) for balancing ventricular volumes. The master controller varies pump flow linearly with heart preload, represented by end-diastolic ventricular pressure. Both master and slave automatically adjust speed at each time step in order to maintain the target flow and inlet pressure, respectively (Stevens, Michael C., et al. “Physiological control of dual rotary pumps as a biventricular assist device using a master/slave approach.” Artificial organs 38.9 (2014): 766-774).

However, these devices rely on the use of cumbersome external sensors, such as pressure, flowrate and/or position sensors, which are invasive to place in the body of the patient and can move over time, leading to inaccurate measurements. Furthermore, their addition to the device can induce extra blood contact with other foreign material, exacerbating the potential for blood damage.

U.S. Pat. No. 8,636,638 discloses a controller for centrifugal BiVAD wherein the RVAD and the LVAD sections share an impeller and wherein said impeller can move axially on magnetic bearings between the cavities of the RVAD and the LVAD. The controller operates by determining a pressure change within at least part of a cavity, which indicates a pressure change in the pulmonary circulatory system and/or the systemic circulatory system, by determining the axial movement of the impeller in response to the force exerted onto it by the pressure change using a position sensor, and by axially moving the impeller in the opposing direction to a balance position, thereby controlling the fluid flow from the RVAD and the LVAD. However, because the RVAD and the LVAD sections share an impeller, the device is bulky and can only be used in one shared operational position. In addition, the control system requires the use of additional mechanical elements in order to operate successfully.

Furthermore, the systems and devices available in the prior art make use of external power sources, such as batteries. The main disadvantage of this is the high risk of infection around the percutaneous drive line between pump and power source. This also has a negative effect on patient mobility and comfort.

WO 2017/217946 belonging to the inventors of the present invention discloses an implantable self-driven pump for use as a cavopulmonary assist device. Said device includes an aortic turbine that uses some systemic blood from the left ventricle as an energy source and a venous pump that is coupled magnetically or mechanically to the turbine. The device operates without need of an external power source.

This type of device can be referred to as an integrated aortic turbine ventricle assist device (iATVA) and can be used as right ventricle support for a right heart failure patient and/or in combination with an LVAD or other available ventricular assist devices.

### SUMMARY

The present invention discloses a biventricular assist system including an LVAD and an iATVA that is able to replicate the Frank-Starling mechanism of the heart passively without need for external sensors or additional mechanisms. The present invention also proposes an alternative control strategy for said biventricular assist system using a master/slave approach. The control strategy is tested to find patient-specific iATVA designs for various right heart failure conditions based on an in silico computational RHF model. Patient-specific iATVA designs can be practically manufactured with 3D printing and the LVAD speed can be adjusted to match the patient's physiologic demand.

There are many advantages of the biventricular assist system of the present invention over prior art. Use of an iATVA allows support of the right ventricle without need of an external energy source, which prevents driveline infections observed in conventional LVADs or BiVADs. The iATVA operates using the systemic blood flow in the aorta, which is driven by an LVAD, therefore establishing a master/slave (MS) relationship between the LVAD and the iATVA. Therefore, the amount of right ventricle support is autoregulated with the MS relationship, providing an improvement on the challenging dual control in conventional BiVADs. The Frank-Starling mechanism of the native heart is directly mimicked by adapting to the aortic flow of the LVAD, and the preload-afterload balance is satisfied through the MS relationship between the LVAD and the iATVA. In addition, deprived filling of left ventricle associated low LVAD output and possible suction events in both ventricles are eliminated due to communication-based increase of iATVA speed and venous return.

### BRIEF DESCRIPTION OF THE DRAWINGS

Accompanying drawings are given solely for the purpose of exemplifying a biventricular assist system, whose advantages over prior art were outlined above and will be explained in brief hereinafter.

The drawings are not meant to delimit the scope of protection as identified in the claims nor should they be referred to alone in an effort to interpret the scope identified in said claims without recourse to the technical disclosure in the description of the present invention.

FIG. 1 demonstrates a biventricular assist system according to the present invention.

FIG. 2 demonstrates a flowchart of the method to fabricate a patient-specific right ventricle support integrated aortic turbine ventricle assist device of the biventricular assist system according to the present invention.

FIG. 3 demonstrates a lumped parameter circuit model of a patient with right heart failure using a biventricular assist system according to the present invention.

FIG. 4 demonstrates sample hemodynamic waveforms of blood pressure in (A) the aorta, (B) inferior vena cava and (C) pulmonary artery with support by a biventricular assist system according to the present invention.

### REFERENCED PARTS LIST

1 Left ventricle assist device (LVAD)
2 Blood turbine
3 Blood pump
4 Heart
5 Aorta
6 Main pulmonary artery
7 Left atrium
8 Right atrium
9 Shaft coupling
10 Integrated aortic turbine ventricle assist device (iATVA)
11 Biventricular assist system

### DETAILED DESCRIPTION OF THE EMBODIMENTS

FIG. 1 illustrates the placement of an embodiment of the present invention, referred to as a biventricular assist system (11). Biventricular assist system (11) includes an LVAD (1) and an iATVA (10) in operational connection with each other. Said iATVA (10) includes a blood turbine (2) operationally configured between the aorta (5) and left atrium (7), and a blood pump (3) operationally configured between the right atrium (8) and main pulmonary artery (6) wherein said blood turbine (2) and blood pump (3) operate synchronously via a coupling (9) between them.

Blood pump (3) is not dependent on an external power source. Rotation required by blood pump (3) is generated by blood turbine (2) and transmitted to blood pump (3) by coupling (9). Coupling (9) may be a mechanical or magnetic coupling. Preferably, coupling (9) is a mechanical coupling, such as a shaft. Coupling (9) can also be made from a flexible material for non-invasive delivery of the entire unit to the body.

LVAD (1) may be any ventricle assist device known in the art that is suitable to provide left ventricle support and having an inflow conduit for receiving blood from a left ventricle of a heart (4) and an outflow conduit for returning a fraction of the blood to blood turbine (2) or the aorta (5), and the rest of the blood to the systemic circulation. The operating conditions of the LVAD (1) can be determined by the surgeons in the early post-operative stage.

Blood turbine (2) may be any device aiming to generate power via the kinetic energy of blood flow. Blood turbine (2) has an inflow conduit suitable for receiving blood directly from said LVAD (1) or from the aorta (5) or a suitable artery having a high blood pressure, and an outflow conduit for returning blood to the systemic circulation. Preferably blood turbine (2) uses a fraction (5-25%, preferably 10-20%, of the overall 5 L/min) of the systemic blood flow that is supplied by the LVAD (1) and transmits this rotation to blood pump (3) via the coupling (9). The fraction of systemic blood used has negligible effect on systemic circulation flowrate and pressure gradient. In a preferred embodiment, blood turbine (2) has a tangential turbine inlet and an axial turbine outlet, from which blood from LVAD (1) is directed to downstream vasculature. Blood flow rotates the turbine impeller and said rotation is transmitted to blood pump (3) via attached shaft (9).

Blood pump (3) has an inflow conduit for receiving blood from a right atrium (8) or right ventricle of a heart (4) and an outflow conduit for returning blood to the pulmonary circulation. Blood pump (3) may be chosen from a variety of pump types, including but not limited to a continuous centrifugal system with rigid or flexible blades, stator, and inlet guide vanes. Blood pump (3) can also be driven by a membrane that is linked to aortic pulsatility. In a preferred embodiment, blood pump (3) is a centrifugal flow pump including a venous pump housing, pump impeller, pump inlet, pump outlet, pump chamber and pump bearing. Deoxygenated blood from right atrium (8) or right ventricle of a heart (4) is conveyed from axial venous pump inlet to tangential venous pump outlet by pump impeller through pump chamber. Pump impeller is rotated by the rotation of said shaft (9). In one embodiment of the invention, the rotational speed varies between 800 to 1800 revolutions per minute, generating a net venous pressure augmentation of 3 to 12 mm Hg through the blood pump (3).

As stated above, blood turbine (2) of iATVA (10) is rotated by the systemic aortic blood flow from the LVAD (1) and transfers the rotation to the blood pump (3) of iATVA (10), which provides right ventricular support. Therefore, the operation of iATVA (10) is dependent on the LVAD (1), creating a MS relationship between LVAD (1) and iATVA (10), which allows autoregulation of the amount of RV support based on a Frank-Starling like mechanism by adapting to the aortic flow. The term “autoregulation” refers to the ability to regulate flow without the need for external sensors or controllers. It was found that use of a left ventricle pump as master resulted in fewer suction events and end-diastolic ventricular pressure compared to using a right ventricle pump as master (Stevens, Michael C., et al. “Physiological control of dual rotary pumps as a biventricular assist device using a master/slave approach.” Artificial organs 38.9 (2014): 766-774). Therefore, use of LVAD (1) as the master and iATVA (10) as the slave is an advantageous configuration.

For example, in the case where LVAD (1) rotation speed increases, blood turbine (2) of the iATVA (10) will also rotate at higher speeds due to the increasing blood turbine (2) inlet flow. Blood turbine (2) and blood pump (3) operate synchronously via the coupling (9) between them and thus they rotate at the same speed. Therefore, the speed of the blood pump (3) and the venous return will increase. Therefore, Frank-Starling law will not be violated and the venous returns in both sides of the heart will be balanced to eliminate the possible left and right ventricle suction events and to maintain stability.

While said biventricular assist system (11) provides a means to autoregulate flow balance, it does not preclude electronic control of the LVAD (1) or iATVA (10). Therefore, in some embodiments, LVAD (1) and/or iATVA (10) may be controlled by an electronic control system. In embodiments where LVAD (1) and/or iATVA (10) are electronically controlled, they may be operated in either continuous or pulsatile mode.

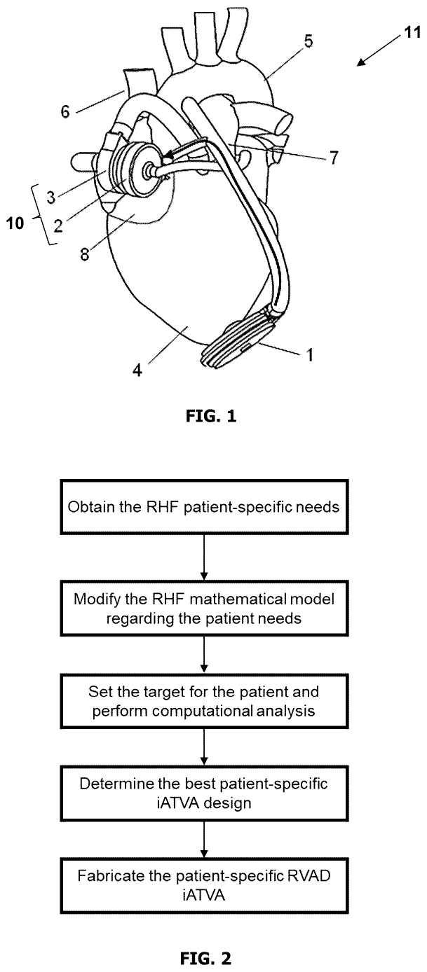

An important aspect in designing ventricular assist devices is matching the device characteristics and dimensions to the relevant patient and disease conditions in order to obtain a device most suitable to the need of the specific patient. FIG. 2 illustrates a flowchart of the method to fabricate a patient-specific right ventricle support iATVA (10). The method includes the following steps:
(i) Obtain the RHF patient-specific hemodynamic parameters needed to design the iATVA. These hemodynamic parameters include systolic/diastolic/average blood pressure in the aorta and pulmonary artery, cardiac output, and the pulmonary blood flowrate.
(ii) Modifying the RHF mathematical model regarding the patient needs.
(iii) Setting the target for the patient and performing computational analysis.
(iv) Determining the best patient-specific iATVA (10) design. This design includes pump and turbine parameters such as the size of the volute, impeller, and blades.
(v) Fabricating the patient specific right ventricle support iATVA (10).

The hemodynamic parameters are largely dependent on the weight and body surface area of the patient and varies among newborn, child, and adult patients. Patients at different age groups will have different hemodynamic parameters. Therefore, there is a need to design the iATVA (10) to patient specifications. For example, a pump with a much higher flowrate will be required for adults with right ventricular failure compared to children. Therefore, the volute, impeller diameter and the impeller blades will need be adjusted accordingly. Parameters may be obtained using magnetic resonance imaging (MRI) and PC-MRI data, and flow and anatomy information may be obtained for sedated patients with institutional review board-approved procedures and informed consent, representing the resting flow conditions and by actual patient catheterization. Preferably, iATVA (10) is fabricated using rapid prototyping methods, such as 3D printing. However, it can be fabricated using any method known in the art.

In one embodiment of the invention, iATVA (10) is in a compressible form, such as a catheter, that can be non-invasively introduced to the patient's body without requiring open heart surgery and can be inflated to its final dimensions in position within the patient.

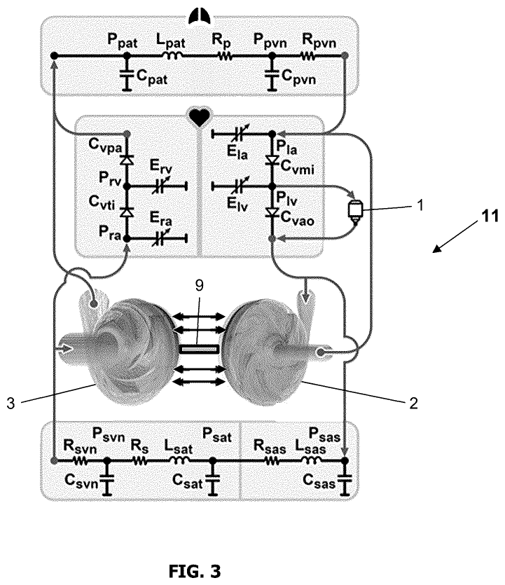

FIG. 3 illustrates a lumped parameter circuit model of a patient with RHF using a biventricular assist system (11). In this model, resistors (R) represent viscous losses in the flow (vascular resistance), inductors (L) represent inertance of the blood, capacitors (C) represent the compliance of the blood vessels and variable capacitors (E) represent the elastance of the heart chamber, as adapted from models described in literature (Avanzolini, Guido, et al. “CADCS simulation of the closed-loop cardiovascular system.” International journal of bio-medical computing 22.1 (1988): 39-49; Suga, Hiroyuki, Kiichi Sagawa, and Artin A. Shoukas. “Load independence of the instantaneous pressure-volume ratio of the canine left ventricle and effects of epinephrine and heart rate on the ratio.” Circulation research 32.3 (1973): 314-322; Stergiopulos, N. I. K. O. S., JEAN-JACQUES Meister, and N. I. C. O. Westerhof. “Determinants of stroke volume and systolic and diastolic aortic pressure.” American Journal of Physiology-Heart and Circulatory Physiology 270.6 (1996): H2050-H2059).

More specifically, within the lungs Pₚₐₜ and Pₚᵥₙ represent the blood pressure in the pulmonary artery and pulmonary vein respectively; Lₚₐₜ represents the inertance of blood in the pulmonary artery; Cₚₐₜ and Cₚᵥₙ represent the compliance of the pulmonary artery and pulmonary vein respectively; and Rₚ, and Rₚᵥₙ represent the pulmonary and pulmonary artery vascular resistance respectively. Within the heart Pᵣᵥ, Pₗᵥ, Pᵣₐ and Pₗₐ and represent the blood pressure in the right and left ventricles and right and left atria respectively; Cᵥₚₐ, Cᵥₜᵢ, Cᵥₘᵢ and Cᵥₐₒ represent the compliance of the pulmonary valve, tricuspid valve, mitral valve, and aortic valve respectively; and Eᵣᵥ, Eₗᵥ, Eᵣₐ and E_{la r}epresent the elastance of the right and left ventricles and right and left atria respectively. And within the body Pₛᵥₙ and Pₛₐₜ and represent the blood pressure in the systemic vein and systemic artery respectively; Cₛᵥₙ and Cₛₐₜ represent the compliance of the systemic vein and systemic artery respectively; Lsat represents the inertance of blood in the systemic artery; and Rₛ and Rₛᵥₙ represent the systemic and systemic vein vascular resistance respectively. The lumped parameter circuit model takes the difference between vascular resistances of the veins and arteries of the pulmonary and systemic circulation into account, therefore iATVA (10) ensures Frank-Starling balance is obtained. Additional lumped parameters may be used to stimulate other diseases a patient might have alongside RHF. In FIG. 3 these are represented by Cₛₐₛ, Lₛₐₛ, Pₛₐₛ and Rₛₐₛ in order to stimulate subaortic stenosis, however, this model can be used to stimulate other diseases by adjusting the compliance, inertance, pressure and resistance values accordingly.

The flowrate of blood between the components of the lumped parameter circuit model are controlled and the pressure and flowrate values for each component are calculated via the differential equation given below:

$$\frac{{d ( CP )}_{i}}{dt} = {\sum }_{j}^{N} \frac{{P}_{j} - {P}_{i}}{{R}_{ji}} + {Q}_{ij , tur} + {Q}_{ij , RVAD} + {Q}_{ij , LVAD}$$

wherein,

i and j: index for each component of the model

C: compliance value of the component

R: resistance value of the component

P: pressure value of the component

Q: flowrate value of the component

Q_{ij,tur}, Q_{ij,RVAD} and Q_{ij,LVAD} represent the flowrate values for the turbine, RVAD and LVAD respectively and are calculated using turbomachine equation and integrated into the lumped parameter circuit model.

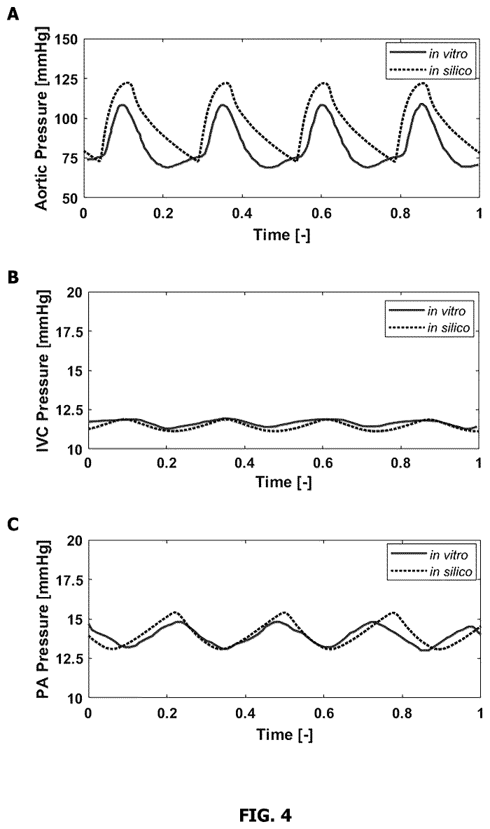

FIG. 4 illustrates the comparison of the hemodynamic waveforms of the blood pressure in the aorta, inferior vena cava and pulmonary artery calculated in silico using the lumped parameter circuit RHF model described above and measured in vitro. In vitro results were obtained using a bench-top single-ventricle mock flow loop system previously described by the inventors with the iATVA (10) of the present invention (Pekkan, Kerem, et al. “In vitro validation of a self-driving aortic-turbine venous-assist device for Fontan patients.” The Journal of thoracic and cardiovascular surgery 156.1 (2018): 292-301; Dur, Onur, et al. “Pulsatile in vitro simulation of the pediatric univentricular circulation for evaluation of cardiopulmonary assist scenarios.” Artificial organs 33.11 (2009): 967-976). It can be seen that the lumped parameter circuit RHF model is successful in simulating the conditions of a RHF patient.

In a nutshell, the present invention proposes a biventricular assist system (11) including a left ventricle assist device (1), an integrated aortic turbine ventricle assist device (10) including a blood pump (3) and a blood turbine (2) coupled by a shaft (9), and a control system.

In one embodiment of the present invention, said LVAD (1) has an inflow conduit configured for receiving blood from a left ventricle of a heart (4) and an outflow conduit configured for returning a fraction of the blood to blood turbine (2) or the aorta (5) and the remaining blood to the systemic circulation, said blood pump (3) has an inflow conduit configured for receiving blood from a right atrium (8) or right ventricle of a heart (4) and an outflow conduit configured for returning blood to the pulmonary circulation and said blood turbine (2) has an inflow conduit configured for receiving blood from said LVAD (1) or from the aorta (5) or an artery having a high blood pressure and an outflow conduit configured for returning blood to the systemic circulation.

In a further embodiment of the present invention, said blood turbine (2) is configured to generate power from the kinetic energy of the blood flow from the outflow from said left ventricle assist device (1) and transmit said power to blood pump (3) via the rotation of said shaft (9) whereby said integrated aortic turbine ventricle assist device (10) is free of an active or external power source to be operable by said blood turbine (2).

In a further embodiment of the present invention, said control system includes left ventricle assist device (1) as a master controller and integrated aortic turbine ventricle assist device (10) as a slave controller whereby the pump speed of blood pump (3) is configured to be variable based on the flowrate of the outflow from said left ventricle assist device (1).

In a further embodiment of the present invention, said integrated aortic turbine ventricle assist device (10) is designed to be patient-specific using right heart failure patient-specific hemodynamic parameters.

In a further embodiment of the present invention, said blood pump (3) is a centrifugal pump.

In a further embodiment of the present invention, said LVAD (1) has an outflow conduit configured for returning a fraction of the blood to blood turbine (2) and said blood turbine (2) has an inflow conduit configured for receiving blood from said LVAD (1).

The present invention also proposes a method for manufacturing a patient-specific integrated aortic turbine ventricle assist device (10) including a blood pump (3) and a blood turbine (2) coupled by a shaft (9), including the steps of:
(i) obtaining the right heart failure patient-specific hemodynamic parameters,
(ii) modifying the right heart failure model with said hemodynamic parameters,
(iii) setting the target for the patient and performing computational analysis,
(iv) determining the required design parameters for the patient-specific integrated aortic turbine ventricle assist device (10), and
(v) fabricating the patient specific right ventricle support integrated aortic turbine ventricle assist device (10) by rapid prototyping.

In a further embodiment of the present invention, said hemodynamic parameters include the systolic, diastolic, and average blood pressure in the aorta and pulmonary artery, the cardiac output, and the pulmonary blood flowrate of the patient.

In a further embodiment of the present invention, said design parameters include the size of the volute, impeller, and impeller blades of the blood pump (3) and blood turbine (2).

The present invention also proposes a patient-specific integrated aortic turbine ventricle assist device (10) produced by said method.

What is claimed is:

1. A biventricular assist system comprising a left ventricle assist device (LVAD), an integrated aortic turbine ventricle assist device comprising a blood pump and a blood turbine coupled by a shaft, and a control system, wherein
the LVAD has an inflow conduit configured for receiving blood from a left ventricle of a heart and an outflow conduit configured for returning a fraction of the blood to blood turbine or an aorta and the remaining blood to a systemic circulation, the blood pump has an inflow conduit configured for receiving blood from a right atrium or right ventricle of the heart and an outflow conduit configured for returning blood to a pulmonary circulation, and said the blood turbine has an inflow conduit configured for receiving blood from the LVAD or from the aorta or an artery having a high blood pressure and an outflow conduit configured for returning blood to the systemic circulation,
the blood turbine is configured to generate power from a kinetic energy of a blood flow from the outflow from the left ventricle assist device and transmit said the power to the blood pump via a rotation of the shaft, wherein said the integrated aortic turbine ventricle assist device is free of an active or external power source to be operable by the blood turbine and
the control system comprises a left ventricle assist device as a master controller and an integrated aortic turbine ventricle assist device as a slave controller, wherein pump speed of the blood pump is configured to be variable based on a flowrate of the outflow from the left ventricle assist device and wherein the control system is passively configured to ensure that Frank-Starling balance is obtained.

2. The biventricular assist system according to claim 1, wherein the integrated aortic turbine ventricle assist device is designed to be patient-specific using right heart failure patient-specific hemodynamic parameters.

3. The biventricular assist system according to claim 1, wherein the blood pump is a centrifugal pump.

4. The biventricular assist system according to claim 1, wherein the LVAD has an the outflow conduit configured for returning a the fraction of the blood to blood turbine and the blood turbine has the inflow conduit configured for receiving the blood from the LVAD.

5. A method for manufacturing a patient-specific integrated aortic turbine ventricle assist device comprising a blood pump and a blood turbine coupled by a shaft comprising steps of:
(vi) obtaining right heart failure patient-specific hemodynamic parameters,
(vii) modifying a right heart failure model with the hemodynamic parameters,
(viii) setting a target for a patient and performing computational analysis,
(ix) determining the required design parameters for the patient-specific integrated aortic turbine ventricle assist device and
(x) fabricating a patient specific right ventricle support integrated aortic turbine ventricle assist device by rapid prototyping.

6. The method according to claim 5, wherein the hemodynamic parameters comprise systolic, diastolic, and average blood pressure in an aorta and pulmonary artery, a cardiac output, and a pulmonary blood flowrate of the patient.

7. The method according to claim 5, wherein the design parameters comprise a size of a volute, impeller, and impeller blades of the blood pump and the blood turbine

8. A patient-specific integrated aortic turbine ventricle assist device produced by the method according to claim 5.

9. The biventricular assist system according to claim 2, wherein the blood pump is a centrifugal pump.

10. The biventricular assist system according to claim 2, wherein the LVAD has the outflow conduit configured for returning the fraction of the blood to blood turbine, and the blood turbine has the inflow conduit configured for receiving the blood from the LVAD.

11. The biventricular assist system according to claim 3, wherein the LVAD has the outflow conduit configured for returning the fraction of the blood to blood turbine, and the blood turbine has the inflow conduit configured for receiving the blood from the LVAD.

12. The method according to claim 6, wherein the design parameters comprise a size of a volute, impeller, and impeller blades of the blood pump and the blood turbine.

13. The patient-specific integrated aortic turbine ventricle assist device according to claim 8, wherein the hemodynamic parameters comprise systolic, diastolic, and average blood pressure in an aorta and pulmonary artery, a cardiac output, and a pulmonary blood flowrate of the patient.

14. The patient-specific integrated aortic turbine ventricle assist device according to claim 8, wherein the design parameters comprise a size of a volute, impeller, and impeller blades of the blood pump and the blood turbine.
